(19) United States
(12) Patent Application Publication
(10) Pub. No.: US 2018/0085262 A1
(43) Pub. Date: Mar. 29, 2018

(54) System and Method for Remote Vapor Detection

(71) Applicant: Shakam LLC, Columbus, OH (US)

(72) Inventors: Jay Schwirian, Monongahela, PA (US); Bradley Yakam, New Albany, OH (US)

(73) Assignee: Shakam LLC, Columbus, OH (US)

(21) Appl. No.: 15/276,362

(22) Filed: Sep. 26, 2016

(51) Int. Cl. A61F 13/42; G06K 19/07

(52) U.S. Cl. CPC A61F 13/42; A61F 2013/424; G06K 19/0723

(57) ABSTRACT

A system for remotely monitoring the status of wetness of a diaper worn by a patient using a reusable electronic vapor sensor.

### FIELD

The current disclosure pertains to systems and methods for remotely monitoring the status of wetness of an incontinence management garment worn by a patient. More particularly, the present disclosure relates to a reusable system for housing a reusable electronic device for use in connection with such a garment.

### BACKGROUND

Urinary incontinence poses many problem issues for patients and staff of nursing facilities, hospitals, hospices, etc. Adult diapers, generally disposable, are utilized to assist in the management of this issue. Changing of the diaper immediately after the incontinence event is considered desirable as it helps in limiting bacterial growth and possible infection. While regularly scheduled diaper changing can help to alleviate such circumstances, it can also result in unnecessary waste of diapers, discomfort to the patient, and expensive use of nursing time.

In accordance therewith, “wired” fluid detection devices have been utilized for many years as detection systems in connection with diapers. However, these systems can be unwieldly and uncomfortable for the patient, may pose risks as to potential entanglement, and can cause issues with respect to cleanliness and reuse.

Most recently, wireless devices, such as RFID tags, have been developed and used. These systems have solved some of the issues related to “wired” systems, but there are still issues. Specifically, with respect to RFID tags, it is noted that there are “passive” and “active” versions thereof.

Passive RFID tags have generated some interest due to the fact that they are relatively inexpensive to produce, and thus can be disposable, and small enough for use in connection with most diaper/garment applications. There have been various attempts to address these issues to date. For example, JP2005000602, entitled “Wet Diaper Informing and Indicating Device and Diaper Equipped with Wet Detecting Device” represents one such attempt. The wet detecting device comprises a passive RFID tag having an IC chip including a data storing medium for storing the number of times that wet signals are generated or the time elapsed since a wet detecting signal was entered, a printed antenna and a communication control section. The control section transmits data stored in the data storing medium to the communication control section via the antenna. The display device comprises a communication control section, a control section, an antenna and a display section. The display section displays, in response to the transmitted data, the number of times that the wet signals are generated or the time elapsed since the wet detecting signal was entered.

However, the use of passive RFID tags in these applications has generally not proved very commercially effective due to a few limiting issues. Specifically, in most cases, the reading range of the passive tags are less than one meter and, sometimes, can only be a few centimeters. This means that the electronic sensor (known as a reader) that is used to receive the data from the passive tag must be positioned very close to the patient thereby requiring each patient to be assigned a separate reader. This can lead to significant expense as passive readers are generally more expensive than active readers, a cost multiplied by the requirement of a separate reader for each individual patient/bed as discussed in JP2005000602.

Accordingly, it would be desirable to utilize a system wherein the sensor is powered and generates its own signal, such as an active RFID. An advantage of the active sensors are that they can transmit data over much larger distances than passive sensors and, given their transmission capabilities, may require of the use of only one, or at least a limited number, of readers. Further, the active sensor readers tend to be less expensive than passive, and the transmission distance may allow patient much more freedom of movement than that of passive tags that require close proximity to a reader to operate properly. Some examples of prior art “active” RFID systems are disclosed in the prior art as well.

For example, U.S. Pat. No. 8,237,572 entitled “Self-powered RFID tag activated by a fluid and method for using such RFID tags” assigned to IBM discloses a system that operates as both an active and a passive RFID tag system. Each RFID tag is attached to an object (or to a structure or a person) on which the presence of a predefined fluid is monitored. In a first state, (absence of the monitored fluid), the tag is acting as a passive RFID tag, and the information it holds, (at least its identifier—ID), can be read with a proximity reader (a hand held or stationary/fixed reader). This operation is performed when the RFID tag is attached to the object (or person) to be monitored. At this stage a table associating tag ID, object name (or person name) and location (or any other information), may be built, and may be recorded so that information it contains is accessible by a control center. In a second state, whenever the monitored fluid appears on the tagged object, a fluid activated battery generates the electrical power which is used to power the RFID tag. The RFID tag then acts as an active RFID tag and starts to emit messages (an alert for instance) which can be received by a distant RFID reader. Alert messages include at least the tag ID but may comprise additional information like a name or a location.

However, despite being operable as an active RFID system, the interaction of urine directly with the tag in the '572 patent means that the tag has to either be thrown out or thoroughly sterilized (in a manner which does not destroy the tag) after every usage. This is expensive, time consuming, or both for a caregiver.

Accordingly, what is desired would be an active sensor device, such as an active RFID sensor, that could be used to sense fluid in a diaper or undergarment wherein the sensor could be mounted or attached to the diaper or garment in a manner in which it can be used and maintained in an uncontaminated manner and wherein it can be detached and reused multiple times.

### SUMMARY

Accordingly, what is provided is a method and system for remotely monitoring the status of wetness of a diaper worn by a patient. More particularly, what is provided is a reusable system for housing a reusable electronic device for use in connection with a diaper.

Specifically, the present disclosure provides a system for remotely monitoring the status of wetness of a diaper worn by a patient using a reusable electronic device wherein the reusable electronic device includes an active, powered transmitter such as an RFID tag and a vapor sensor.

More specifically, the present disclosure may comprise a pouch portion located on the outer surface of a disposable diaper. The pouch portion may be comprised of an outer layer that is fluid and vapor impermeable and inner layer that is sealed to the outer layer of the disposable diaper that is vapor permeable, fluid impermeable. The pouch portion may include an electronic vapor sensor therein attached to a powered transmitter, such as an active RFID tag. The outer surface of the diaper may have holes or slits cut therethrough where the inner layer of the pouch is attached thereover. In such an embodiment, the vapor permeable, fluid impermeable barrier maybe material such as Gore Tex™, and may cover the holes/slits such that vapor ingress into the pouch portion is permitted while fluid ingress into the pouch portion is prevented. Other aspects and advantages of the disclosure may be apparent to those of ordinary skill in the art upon review of the description, drawings and claims of the present disclosure and which would be considered within the scope of the disclosure.

### BRIEF DESCRIPTION OF THE DRAWINGS

FIG. 1 is a front perspective view of a incontinence management system including a pouch for housing a vapor sensor therein in accordance with aspects of the present disclosure;

FIG. 2. is a front perspective view of an alternate embodiment of the incontinence management system of FIG. 1;

FIG. 3 is a top elevation view of the pouch portion shown in FIG. 1; and

FIG. 4 is a front plan view of the pouch of FIG. 3.

### DETAILED DESCRIPTION

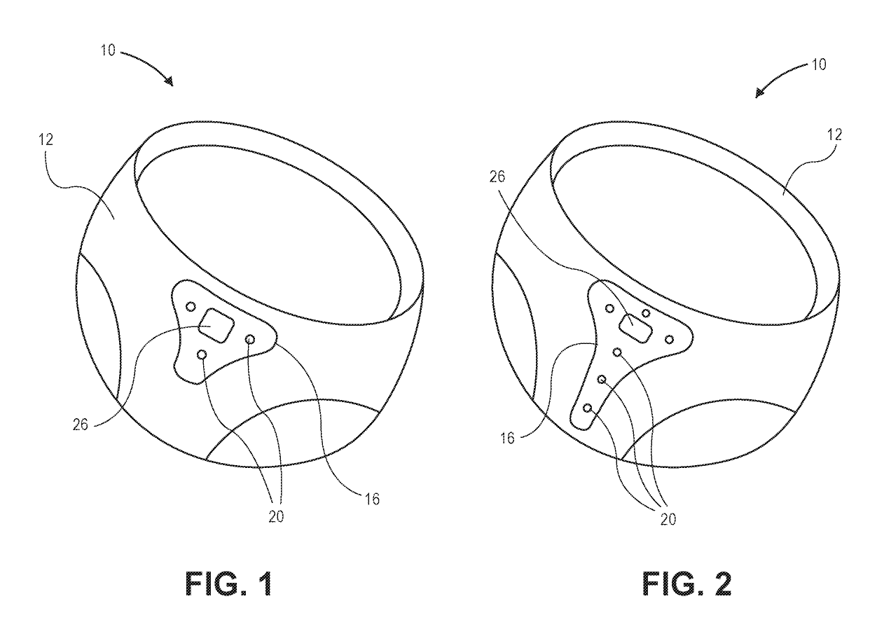

The present invention will be described with reference to the accompanying figures. where like reference numbers correspond to like elements. As shown best in FIGS. 1 and 2, the present disclosure provides an incontinence management system 10 comprising a disposable diaper 12 having a pouch portion 14 attached thereto.

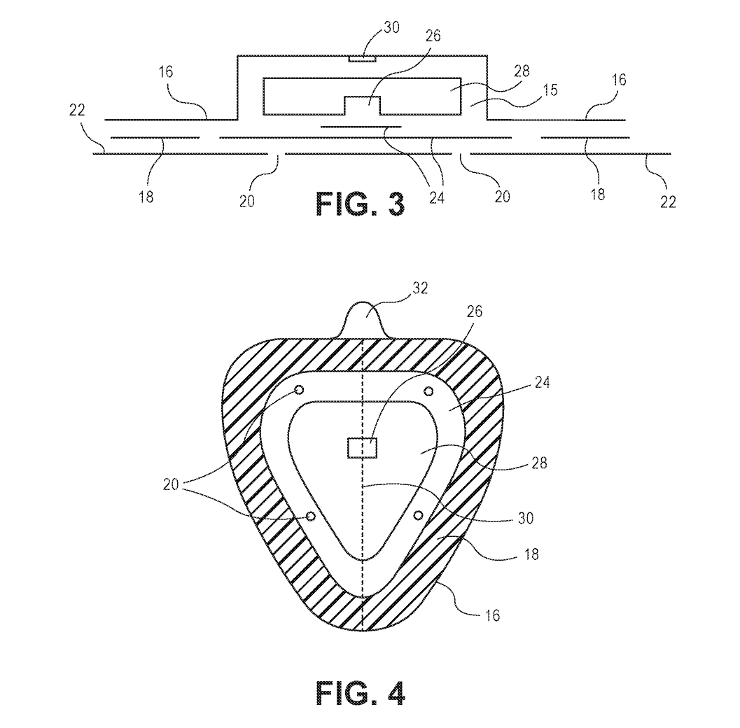

As best shown in FIGS. 3 and 4, the outer layer 16 of the pouch portion 14 is preferably made from a fluid impermeable material, such as polyurethane, and may be attached to the outer surface 22 of the diaper 12 by adhesive 18 (or the like) or any other means, such as heat sealing, whereby a fluid impermeable barrier is formed between the outer layer of the diaper 12 and the inner portion 15 of the pouch portion 14. Fluid passages 20, such as holes or slits, may be provided in the outside layer 22 of the diaper 12 allowing vapor from an inside area of the diaper 12 to be received into the interior portion 15 of the pouch portion 14. In some embodiments, as shown in FIG. 1 and FIG. 2, the pouch portion 14 may be shaped in different ways, and the holes 20 may be positioned strategically based upon the age and/or gender of the system 10 wearer.

In accordance with an embodiment of the disclosure, a vapor permeable, fluid impermeable liner 24, such as Gore Tex™ (i.e. stretchable fabric including PTFE), is placed over all of the holes/slits 20 such that vapor egress into the interior portion 15 of the pouch portion 14 is permitted while fluid egress into the interior portion 15 of the pouch portion 14 is prevented. A vapor sensor 26, which is electrically connected to an electronic transmitter 28, such as an active RFID tag, may be removably attached to the fluid impermeable liner 24 or the inside of the outer layer 16 of the pouch portion 14. In accordance with some embodiments of the disclosure, the vapor sensor 26 may be positioned a distance from the holes 20 allowing the diaper's 12 outer layer 22 and the pouch portion's 14 outer layer 16 to act as a conduit for vapor. The outer layer 16 of the pouch portion 14 is preferably provided with a fluid impermeable mechanism 30 for easy opening thereof so that a caregiver may remove the vapor sensor 26 and active electronic transmitter 28 from the pouch portion 14 following use thereof by a patient prior to disposal of the rest of the system 10. The mechanism 30 may be perforations or a “rip zip” style opening that is opened by a caregiver grabbing pull tab 32 and pulling downward.

In operation, in accordance with an embodiment of the disclosure, a user may wear the system 10 of the present disclosure. Upon the occurrence of an incontinent event, the vapor sensor 26 may detect the presence of a certain predetermined amount of vapor as is known in the art. The sensor 26 then sends a signal via the transmitter 28 to a reader (not shown) which indicates to the caregiver that the wearer's diaper 12 has been wet. The caregiver may then remove the garment system 10 from the wearer and remove for reuse the vapor sensor 26 and transmitter 28 while disposing of the diaper 12.

While example embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the express scope of the following claims. Likewise, it is to be understood that it is not necessary to meet any or all of the stated advantages or objects disclosed herein to fall within the scope of the disclosure, since inherent or unforeseen advantages may exist even though they may not have been explicitly discussed herein.

What is claimed is:

1. A pouch for containing a sensor for use in connection with an incontinence management garment comprising:
a pouch portion attachable to an incontinence management garment, the pouch creating an interior portion;
a vapor sensor positioned in the interior portion of the pouch and configured to sense a vapor percentage reading;
wherein the pouch is comprised of an outer layer of a fluid impermeable material and an inner layer of a vapor permeable, fluid impermeable material.

2. The pouch of claim 1 further comprising a transmitter operably connected to the vapor sensor.

3. The pouch of claim 3 wherein the transmitter is an RFID tag.

4. The pouch of claim 2 wherein the RFID tag is an active RFID tag.

5. The pouch of claim 1 wherein the inner layer is comprised of a fabric that includes PTFE incorporated therein.

6. The pouch of claim 1 wherein the pouch is removably attachable to an incontinence garment.

7. The pouch of claim 1 wherein the outer layer includes a fluid impermeable opening mechanism incorporated therein.

8. The pouch of claim 7 wherein the opening mechanism comprises perforations.

9. The pouch of claim 1 wherein the pouch is integrally incorporated in an incontinence management garment.

10. The pouch of claim 9 wherein the incontinence management garment is a diaper.

11. An incontinence management system comprising;
a diaper having an outer surface having openings therein for allowing a vapor therethrough;
a pouch portion attached to the diaper over the openings in the outer surface of the diaper, the pouch creating an interior portion;
a vapor sensor positioned in the interior portion of the pouch and configured to sense a vapor percentage reading;
wherein the pouch is comprised of an outer layer of a fluid impermeable material and an inner layer of a vapor permeable, fluid impermeable material.

12. The incontinence management system of claim 11 further comprising a transmitter operably connected to the vapor sensor.

13. The incontinence management system of claim 12 wherein the transmitter is an RFID tag.

14. The incontinence management system of claim 13 wherein the RFID tag is an active RFID tag.

15. The incontinence management system of claim 11 wherein the inner layer is comprised of a fabric that includes PTFE incorporated therein.

16. The incontinence management system of claim 11 wherein the pouch is removably attachable to the diaper.

17. The incontinence management system of claim 11 wherein the outer layer includes a fluid impermeable opening mechanism incorporated therein.

18. An incontinence management system comprising;
a diaper having an outer surface having openings therein for allowing a vapor therethrough;
a pouch portion integrally incorporated into the diaper over the openings in the outer surface of the diaper, the pouch creating an interior portion;
a vapor sensor positioned in the interior portion of the pouch and configured to sense a vapor percentage reading;
wherein the pouch is comprised of an outer layer of a fluid impermeable material and an inner layer of a vapor permeable, fluid impermeable material and the pouch is attached to the diaper such that a fluid impermeable barrier is created between the inner layer of the pouch and the outer surface of the diaper that covers the openings.

19. The incontinence management system of claim 18 further comprising an active RFID tag operably connected to the vapor sensor.

20. The incontinence management system of claim 18 wherein the inner layer is comprised of a fabric that includes PTFE incorporated therein.
